(19) United States
(12) Patent Application Publication
(10) Pub. No.: US 2011/0220125 A1
(43) Pub. Date: Sep. 15, 2011

(54) MANDIBULAR ADVANCEMENT SPLINT

(75) Inventors: Mark Van Dyke, Fairview, PA (US); John H. Tucker, Erie, PA (US)

(73) Assignee: Advanced Airway Design LLC

(21) Appl. No.: 13/048,091

(22) Filed: Mar. 15, 2011

Related U.S. Application Data
(60) Provisional application No. 61/313,966, filed on Mar. 15, 2010.

(51) Int. Cl. A61F 5/56

(52) U.S. Cl. 128/848

(57) ABSTRACT

A mandibular advancement splint provides for adjustable lateral movement of upper and lower appliances. An upper appliance is inserted onto a user's upper jaw and a lower appliance is inserted onto a user's lower jaw. A first protrusion is formed on the upper appliance. A second protrusion is formed on the lower appliance. One of the first protrusion and the second protrusion can be adjusted to offset the lower jaw with respect to the upper jaw.

The splint can have an upper appliance having a body mounted to an upper jaw of a user; and a lower appliance having a body mounted to a lower jaw of a user. The upper appliance has a pair of adjustable wings attached to the upper appliance body; and the lower appliance has a pair of fixed wings attached to the lower appliance body. The pair of wings attached to said upper appliance are slidably adjustable with respect to the upper appliance body to determine the offset between the upper and lower jaws of the user.

### CLAIM OF PRIORITY

This application claims priority from U.S. Provisional Patent Application Ser. No. 61/313,966 filed on Mar. 15, 2010, which is hereby incorporated by reference.

### BACKGROUND OF THE DISCLOSURE

This disclosure relates generally to medical and dental devices, and more particularly to an apparatus for prevention of snoring and improved breathing during sleep. This disclosure relates to a mandibular advancement device that has application in the treatment of orthodontic conditions, snoring, obstructive sleep apnea (OSA) and certain temporomandibular joint disorders.

Snoring is a problem that plagues millions of people. It is generally thought that snoring and OSA occur when there is at least partial occlusion of the airway and that the tongue is involved in this. Snoring and OSA commonly occur during sleep. Mandibular advancement devices advance the lower jaw carrying the tongue forward thereby reducing the likelihood of the tongue impacting on the airway.

Numerous forms of mandibular advancement devices are known. One example is a mandible repositioning appliance formed by an upper bite block and a lower bite block interconnected by an extendible connector. The arms that join the lower and upper bite blocks extend from a location proximate the lower incisors rearwardly at an inclined angle, to be anchored in the roof of the mouth. A disadvantage with this arrangement is the connector and attachment arms intrude excessively into the oral cavity, and the resulting interference may limit efficacy and/or it may be progressively less effective with increasing mouth opening, or it may not permit jaw opening. The connectors embedded in the lower bit block and the limitation to jaw closure may also limit compliance.

Mandibular advancement represents locating of the mandible so that it functions in the protruded range from the reflex or habitual path of closure (occurring between the intercuspal occlusal position and the maximum open position) to the protrusive border path.

Consequently, many attempts have been made to solve the snoring problem. One known technique is to extend the lower jaw of a snorer forwardly thereby opening the air passage and reducing or preventing snoring.

The amount of forward extension of the lower jaw that is most effective at reducing or eliminating snoring varies from person to person. In existing art devices, the amount of forward adjustment is fixed, and thus cannot be adjusted by the user to increase the effectiveness of the device or to increase comfort. Therefore, a need has arisen for a device that allows for adjustment of the forward extension of the lower jaw in an anti-snoring device. Also, existing devices do not allow independent lateral movement of the upper and lower appliances.

Thus, there is a need for a dental device which allows independent and adjustable lateral movement of the upper and lower devices which overcome the above-mentioned deficiencies while providing improved overall results.

### SUMMARY OF THE DISCLOSURE

The present disclosure relates to a dental device for prevention of snoring and improves breathing during sleep. More particularly, the disclosure provides a mandibular advancement device for the treatment of Obstructive Sleep Apnea and/or snoring including a lower appliance having an attachment structure adapted to be releasably attachable to at least a portion of the lower jaw and an engagement surface extending upwardly from the appliance and an upper appliance having an attachment structure adapted to be releasably attachable to at least a portion of the upper jaw and an engagement surface extending downwardly from said attachment structure; and wherein, when fitted to a patient, the lower and upper appliances are adapted to engage at a location lying in an area beside and close to the posterior teeth in a manner to cause advancement of the lower jaw from the reflex path of opening and to maintain the engagement and advancement, while permitting sagittal movement, up to the normal range of jaw opening extending from an advanced occluding position.

It is an object of the present disclosure to provide a mandibular advancement device that provides advancement of the lower jaw, and permits freedom of jaw movement (i.e., jaw opening) while retaining advancement within a range protruded from the reflex or habitual path of closure.

It is a further object of the disclosure to provide a mandibular advancement device which can permit closure to the protruded occusal position.

It is a further object of the disclosure to provide a mandibular advancement device which can be adjustable to give a variable extend of advancement of the lower jaw.

It is a further object of the disclosure to provide a mandibular advancement device having minimal interference with the tongue, the oral airway, mouth seal and the fundamental tongue space.

In accordance with one aspect of the disclosure, a mandibular advancement splint provides for adjustable lateral movement of upper and lower appliances. The splint includes an upper appliance which is inserted onto a user's upper jaw; a lower appliance which is inserted onto a user's lower jaw; a first protrusion formed on the upper appliance; and a second protrusion formed on the lower appliance, wherein one of the first protrusion and the second protrusion can be adjusted to offset the lower jaw with respect to the upper jaw.

In accordance with another aspect of the disclosure, a mandibular advancement splint has an upper appliance having a body mounted to an upper jaw of a user; and a lower appliance having a body mounted to a lower jaw of a user. The upper appliance has a pair of wings attached to the upper appliance body. The lower appliance has a pair of wings attached to the lower appliance body. The pair of wings attached to the upper appliance are slidably adjustable with respect to the upper appliance body to determine the offset between the upper and lower jaws.

In accordance with another aspect of the present disclosure, a dental device is provided which substantially eliminates or reduces disadvantages and problems associated with existing anti-snoring devices. In particular, a dental device is provided in which an upper appliance fits the user's upper teeth, and a lower appliance fits the user's lower teeth. A post or “fang” extends upwardly from the lower appliance, and the fang includes an aperture or slot for receiving a shim and causes the user's lower jaw to extend forward with respect to the user. The position and thickness of the shim determine the amount of forward extension of the user's lower jaw.

The upper appliance has a flat surface formed on the appliance or a protrusion attached to the appliance which matingly engages the fang of the lower appliance. Another aspect of the disclosure is that the upper and lower appliances allow the upper and lower jaws to independently move laterally from side to side and vertically with respect to each other.

In accordance with another aspect of the disclosure, the shim is removable and adjustable.

In accordance with another aspect of the disclosure, an air supply tube is connected to the upper and lower appliance to provide supplemental forced air to the patient to facilitate air intake.

In accordance with another aspect of the disclosure, the fang has a gap to accommodate an air supply tube.

In accordance with another aspect of the present disclosure, the upper and lower appliances are temporary appliances which include an acrylic or plastic tray which holds a liner for an individualized fit.

In accordance with another aspect of the disclosure, a custom fit oral appliance is made directly on a mold of the patient's upper and lower teeth.

In accordance with another aspect of the disclosure, an appliance is used for sports and/or concussions including bite material and an energy absorbing material.

In accordance with another aspect of the disclosure, wings are added to the upper and lower appliances, wherein the lower appliance wings are fixed and the upper appliance wings are slidably adjustable to determine the amount of offset of the jaws.

Other aspects of the disclosure will become apparent upon a reading and understanding of the following detailed description.

### BRIEF DESCRIPTION OF THE DRAWINGS

Embodiments of the invention now will be described with reference to the accompanying drawings, in which,

FIG. 1 shows a side elevational view of the human skull with a device attached to the upper and lower jaws in accordance with a preferred embodiment of the present disclosure;

FIG. 1A is a front elevational view of a lower fang of FIG. 1;

FIG. 1B is a top plan view of the lower fang of FIG. 1A;

FIG. 2 is a side elevational view of the lower appliance attached to the bottom jaw;

FIG. 3 is a side elevational view of the upper appliance attached to the upper jaw;

FIG. 4A is a top plan view of the lower appliance attached to the lower jaw;

FIG. 4B is a top plan view of the upper appliance attached to the upper jaw;

FIG. 5 is a side elevational view of a human skull with an upper and lower appliance having air supply connectors in accordance with another embodiment of the present disclosure;

FIG. 5A is a front elevational view of the fang with a channel formed therein to accommodate the air supply connector;

FIG. 5B is a top plan view of an air supply connector attached to the upper or lower appliance and an air supply tube extending around the upper or lower appliance;

FIG. 5C is a rear elevational view of the upper or lower appliance with the air supply connector attached (right side);

FIG. 5D is a rear elevational view of the upper or lower appliance with the air supply connector attached (left side);

FIG. 6 is an elevational end view of a temporary appliance in accordance with another embodiment of the disclosure;

FIG. 7 is an elevational end view of a custom fit appliance in accordance with another aspect of the disclosure;

FIG. 8 is an elevational end view of a sports appliance in accordance with another embodiment of the present disclosure;

FIG. 9 is an elevational end view of a sports concussion appliance in accordance with another embodiment of the present disclosure;

FIG. 10 shows a side elevational view of a human skull with an upper appliance mounted to an upper jaw and a lower jaw wherein the upper appliance has adjustable wings and the lower appliance has fixed wings in accordance with another aspect of the disclosure;

FIG. 11 is a top plan view of the upper appliance of FIG. 10;

FIG. 11A is a front elevational view of the upper appliance of FIG. 10;

FIG. 11B is a side elevational view of the upper appliance of FIG. 10;

FIG. 12 is a top plan view of the lower appliance of FIG. 10;

FIG. 12A is a front elevational view of the lower appliance of FIG. 10; and

FIG. 12B is a side elevational view of the lower appliance of FIG. 10.

### DETAILED DESCRIPTION OF THE DISCLOSURE

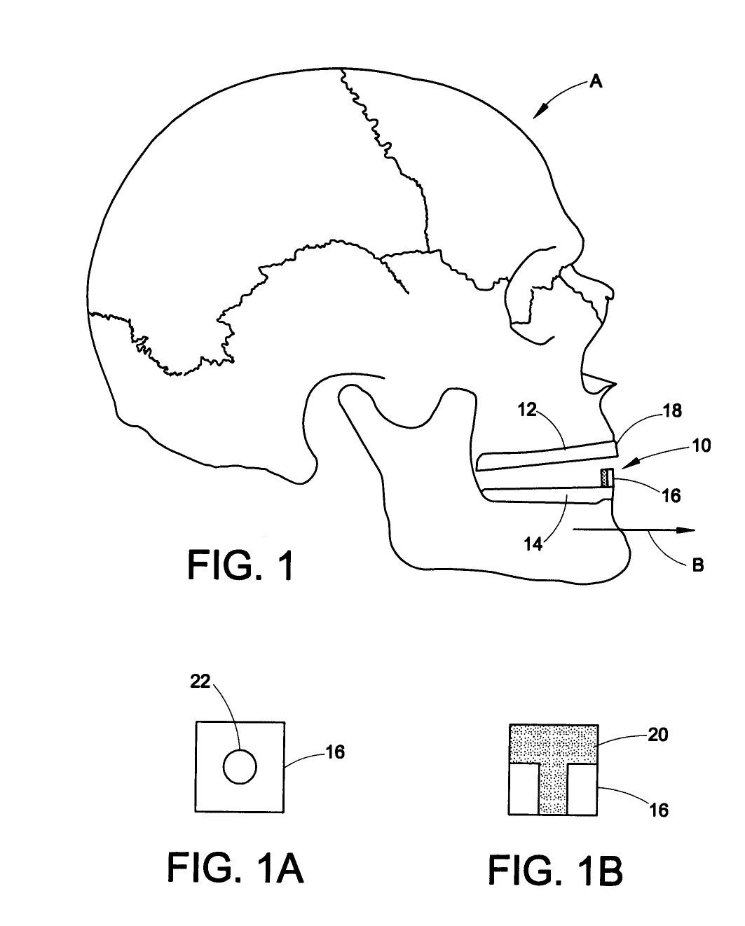

FIG. 1 illustrates a side elevational view of a human skull A with a mandibular advancement device 10 constructed according to a preferred embodiment of the present invention. In FIG. 1, the normal bite (occlusal) position for the teeth is shown, and particularly the relationship between the upper jaw and the lower jaw. In performing mandibular advancement treatment, it is desired to advance a lower jaw to a position relative to the upper jaw. The degree of advancement can depend upon clinical requirements. The relative displacement of the hinge point can be seen to have both horizontal and vertical components. Advancement of the lower jaw carries the tongue forward so that (particularly in sleep) there is a greatly reduced tendency for the tongue to impinge on the pharynx. The degree of advancement can be from the reflex or habitual closing path to the anterior border path.

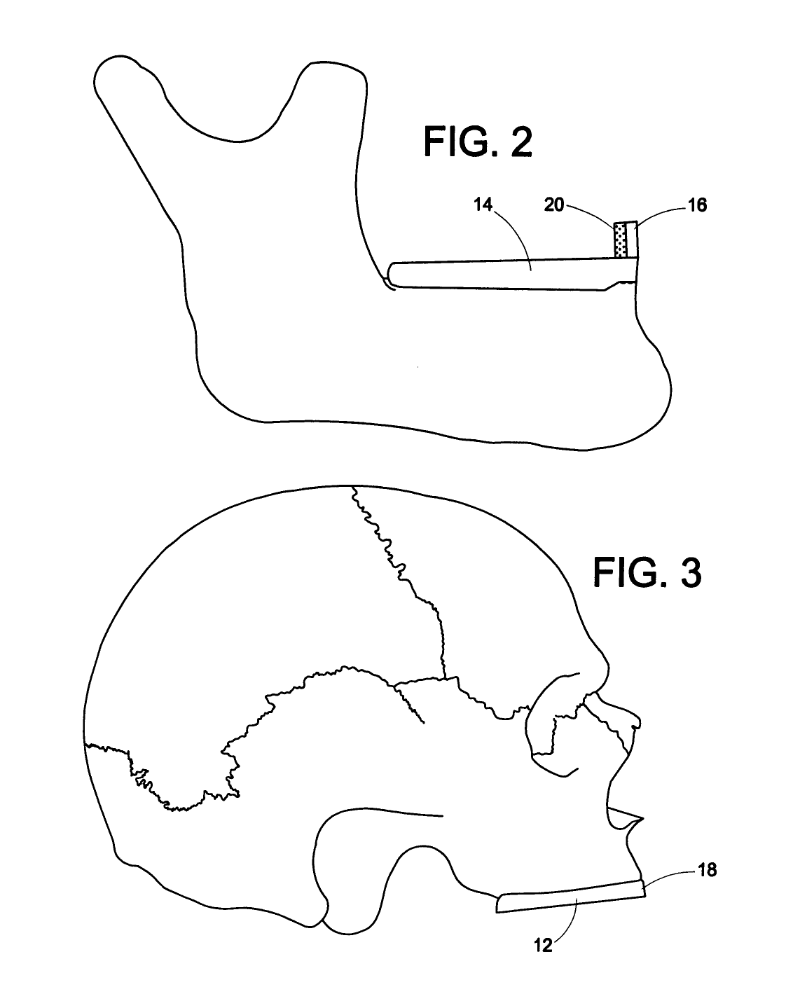

As shown in FIGS. 1, 2 and 3, device 10 includes an upper appliance 12 and a lower appliance 14 which are fitted onto the upper and lower jaws. Appliance 12, 14 may be made from any material suitable for dental uses, such as a hard acrylic material, or methylmethacrylate or a polycarbonate resin thermoplastic. Such materials are known to those familiar with dental mouthpieces, and other materials may be used without departing from the intended scope herein.

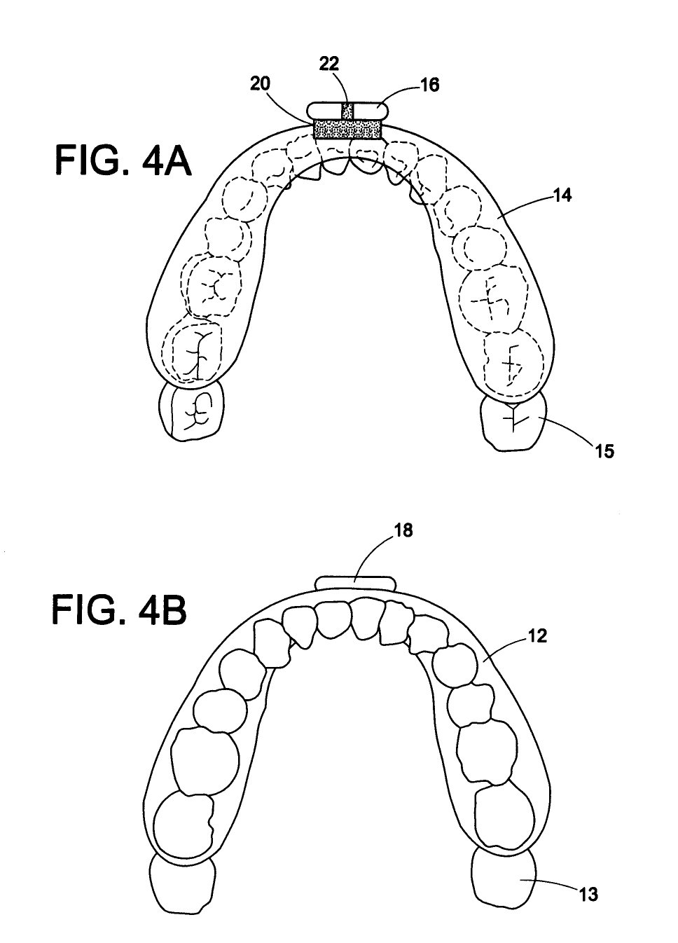

Appliances 12 and 14 fit at least some of a user's upper and lower arches of teeth 13, 15, respectively (FIGS. 4A, 4B). Extending upwardly from lower appliance 14 is a post or “fang” 16. Post 16 is shown as square-shaped, but other configurations are contemplated by the disclosure. Post 16, when in use, makes contact with a flat mating surface 18 of upper appliance 12. This contact between post 16 and surface 18 causes lower appliance 14, and consequently a user's lower jaw, to extend slightly forward (to the right in FIG. 1) in the direction of arrow B in FIG. 1. Forward extension of the lower jaw allows the air passage of the user to remain open, thereby preventing or minimizing snoring and improving breathing during sleep.

Post 16 may have a wide range of shapes, including various lengths, depths, and widths, to perform this function. The term “post” or “fang” is used to describe any such structure. Furthermore, post 16 may directly contact the upper appliance 12, or it may contact an attachment to upper appliance 12, such as a tab, an adjustable tab, any attachment extending upward, or any other attachment to upper appliance 12 which forms the flat surface 18.

Referring to FIGS. 1A and 1B, the post 16 preferably has a removable and adjustable shim 20 which is inserted into an opening 22 found in the fang. The shim is shown as T-shaped, but can have other configurations without departing from the scope of the disclosure. The shim thickness is adjustable so as to allow adjustment of the amount of forward extension of the user's lower jaw. The shim may be made of the same material as the appliance 14, or any suitable dental material.

Referring to FIGS. 4A and 4B, upper appliance 12 has flat protruding surface 18 which matingly engages the post 16. Surface 18 may be integral to the appliance 12 or it may be formed by a separate tab or post as seen in FIG. 4B. Post or fang 16 can slide along the surface 18 which in turn moves the fang and the lower appliance and lower jaw forward.

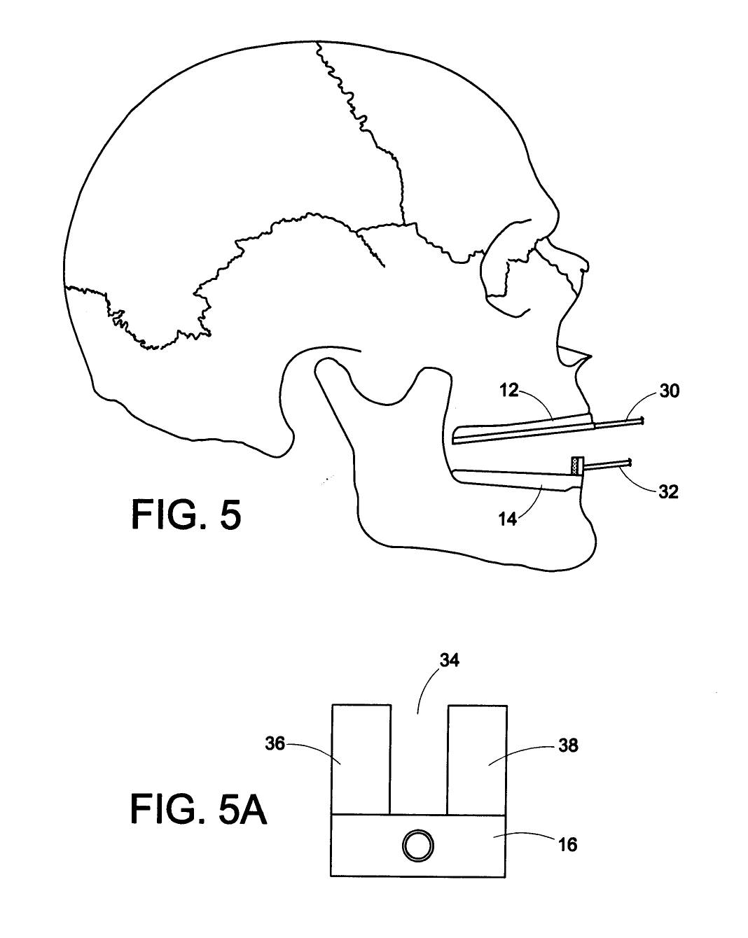

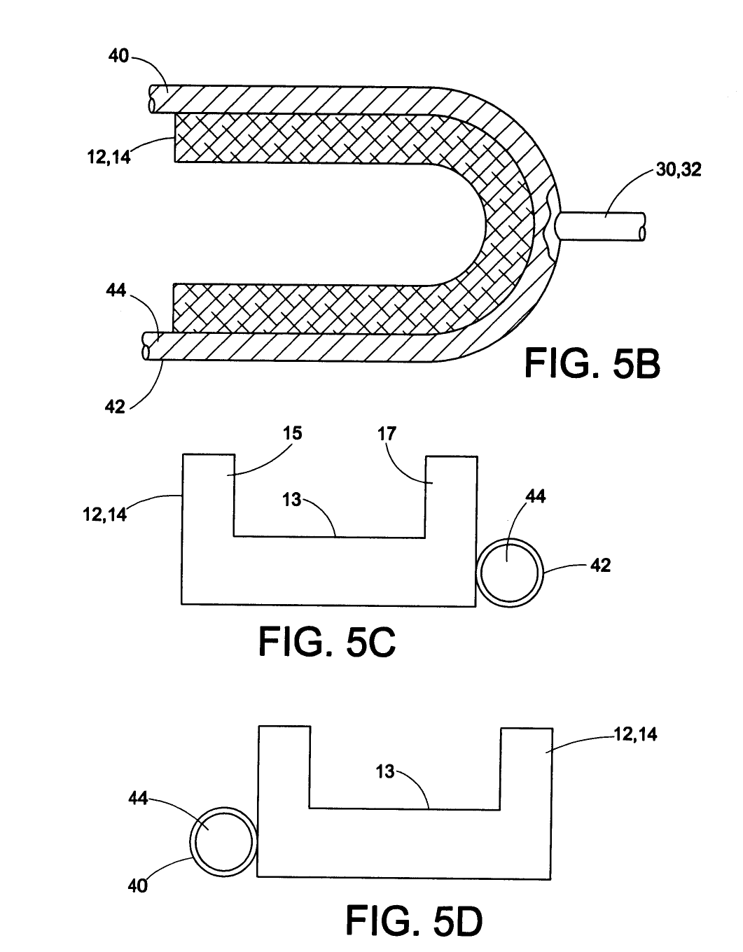

Referring now to FIGS. 5, 5A, 5B, 5C and 5D, in accordance with another aspect of the disclosure, an air supply tube or connector 30, 32 is provided to upper and lower appliances 12, 14, respectively, to provide continuous or intermittent supply of air into the patient's mouth. Specifically, referring to FIG. 5A, the post or fang 16 has a gap 34 formed between parallel walls 36, 38 for accommodating the air supply connector 32 therebetween.

Referring to FIGS. 5B, 5C and 5D, airway tubes 40, 42 are formed on opposite right and left sides of the upper and lower appliances 12, 14 which extend around the perimeter of both sides of the upper and lower appliances to accommodate an air supply which passes through opening 44.

Referring to FIGS. 5C and 5D, a channel 13 is formed in upper appliance 12 or lower appliance 14 via walls 15, 17 to accommodate deformable material or the user's teeth therein.

The air supply connector connects to an air supply such as a BPAP (Biaural Positive Airway Pressure) or PAP (Positive Airway Pressure) machine (not shown) and the air supply tube 40, 42 carries high pressure air to the oropharynx or the back of the throat. The air can be intermittently supplied and may be activated when the patient takes a breath. The airway channels can be custom fitted or airway advanced.

Some existing appliances are used with a CPAP machine, which has the disadvantage of a constant air supply being provided, which may not be comfortable or desirable for the patient.

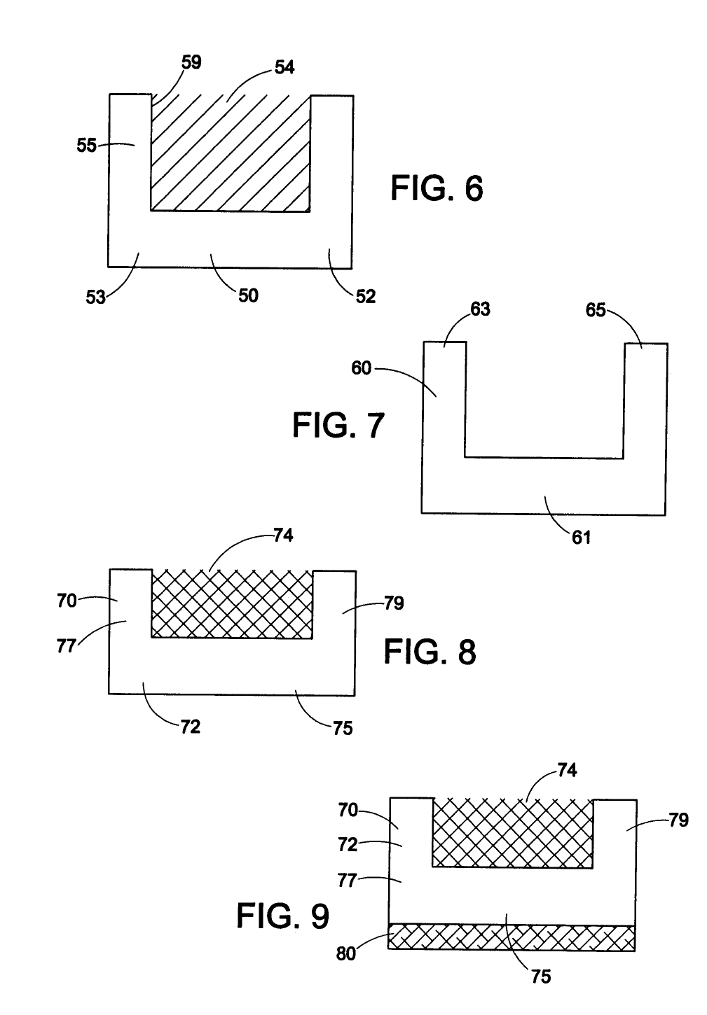

FIGS. 6-9 illustrate various modifications to the appliances previously discussed and shown in FIGS. 1-4B. The discussion regarding FIGS. 1-4B may also be applied to FIGS. 6-9.

Referring to FIG. 6, in accordance with another embodiment of the disclosure, a temporary appliance 50 is provided. The appliance 50 has a similar appearance as the upper and lower appliances in FIGS. 4A and 4B.

An acrylic or plastic tray 52 having a lower end 53 and two parallel side walls 53, 57 is used to hold a liner or a dental impression or deformable material 54, which is used for an individualized or customized fit. An advantage of this device is that it is low cost and is easily adjustable to each patient. The appliance can also be made virtually next to the patient's chair as they wait.

Deformable material 54 is bonded to the inner area 59 of the tray 52 and used for custom forming of a mold of the user's teeth for proper fitting during use. By using deformable material, each user can customize his or her anti-snoring device without the expense associated with having a dental mold prepared by a dental professional.

A suitable material for deformable material 54 is an ethylene-vinyl acetate copolymer resin. Any other suitable deformable materials may also be used. Typically, with such material, the material is heated to a temperature of about 150 Fahrenheit, through a microwave oven or by heating in hot water, for example, so as to place the material in its deformable state. A user then inserts the appliance and bites down, thereby deforming the material into the shape of the user's upper arch of teeth. The appliance is then removed and allowed to cool, thereby setting the material into a mold of the user's upper arch.

Likewise, lower appliance includes a tray filled with a deformable material. A mold of the lower arch of teeth is formed as described above for FIG. 6 in connection with upper arch.

Referring to FIG. 7, in accordance with another embodiment of the disclosure, a custom fit appliance tray 60 made of acrylic or plastic material is made directly on a model of the patient's upper and lower jaws. Again, the tray 60 has a lower portion 61 and two parallel side walls 63, 65 would have a similar appearance as the appliances described in FIGS. 1-4B.

Referring to FIG. 8, in accordance with another embodiment of the disclosure, an appliance 70 is used for a sports application. The appliance has a plastic or acrylic shell 72, and a boil or bite impression material 74 therein between lower portions 75 and two parallel side walls 77, 79. During sports performance, such as in running or jogging, airway resistance may occur. The appliance helps open the airway at least on a temporary basis to increase airway performance. Again, the appliance 70 would have a similar appearance as the appliances described in FIGS. 1-4B.

Referring to FIG. 9, in accordance with another embodiment of the disclosure, a viscoelastic energy absorbing material 80 is supplied on each side of the patient's teeth to have absorptive properties and anti-concussive properties by providing a gap in the patient's jaws. The absorbing material 80 can be used in conjunction with any of the appliances described herein.

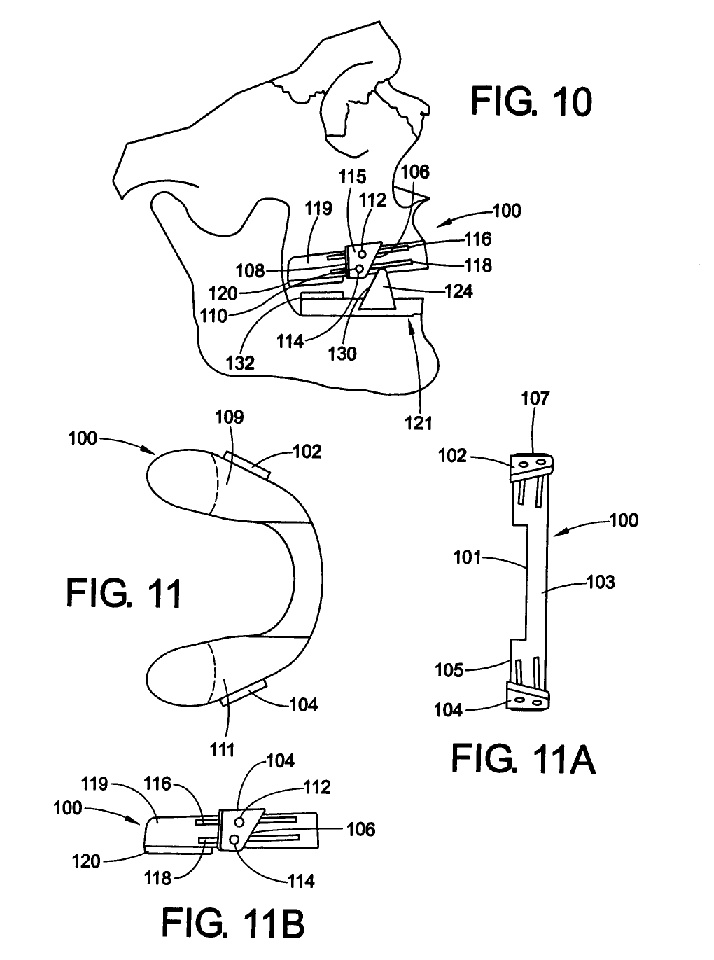

Referring now to FIGS. 10-12, an alternate embodiment of the disclosure is shown. Upper appliance body 100 has a U-shaped channel 101 formed in a central portion 103 thereof. Referring to FIG. 11A, central portion 103 has a thinner cross section than outer lateral portions 105, 107 to facilitate placement of moldable material on an upper portion of the appliance or the user's teeth and has a pair of adjustable wings or engaging members 102, 104 which are slidably attached to opposed walls 109, 111 of upper appliance 100. Wings 102, 104 have an angled front wall 106, having an acute angle of about 30 degrees or 45 degrees from horizontal and a substantially straight rear wall 108. Several screws or other fasteners 110 are threaded through holes 112, 114 in side walls 115 of the wings. A pair of elongated lateral grooves or channels 116, 118 is formed in opposed side walls 119 of the upper appliance 100. Wings 102, 104 can slide laterally (to the left or right in FIGS. 10 and 11B) along channels 116, 118 and is secured to the preferred position via screws 110 which extend into channels 116, 118.

Added material or shim 120 is placed or mounted at an underside of the appliance body 100 at a rear or posterior portion of the body to provide more of a vertical gap between the appliances and to allow the tongue to more easily move forwardly. Preferably the shim has a height of about 2 millimeters (0.0002). However, other thicknesses are contemplated by the disclosure.

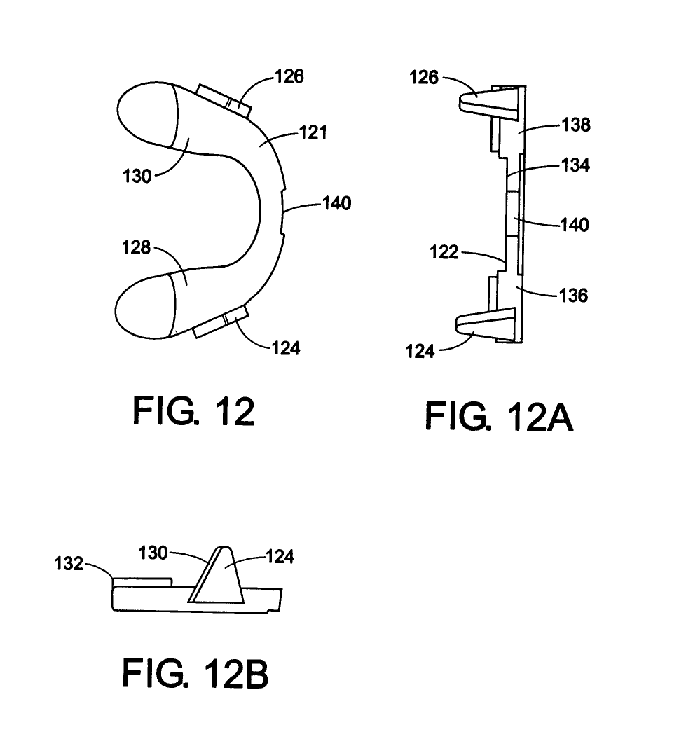

Referring to FIGS. 12-12B, the lower appliance has a body 121 having a substantially U-shaped channel 122 formed in a central portion 134 of the body. A pair of laterally opposed wings or protrusions 124, 126 are permanently or fixedly attached to opposite side walls 128, 130 of body 121. Alternatively, the wings can be integrally formed as part of the body 121 and are contiguous with body 121. The wings 124, 126 each has an angled rear wall 130 (having an obtuse angle of about 150 degrees or 135 degrees from horizontal) which matingly engages the angled front wall 106 of the movable upper wings 102, 104. An additional amount of solid material or shim 132 is added or stacked onto an upper rear portion of body 121 to facilitate movement of the tongue forwardly. The stack or shim can be about 2 millimeters in height or so on the body for the back teeth to enable the tongue to easily slide forward.

Referring to the front view in FIG. 12A, central portion 134 of the body has a thinner cross section than the outer portions 136, 138 to facilitate placement of moldable material. There is also a notch 140 formed in the central area to receive and hold a measurement device (not shown).

Thus, the upper appliance wings are adjustably and slidably positioned to a desired location to result in a desired offset of the lower jaw with respect to the upper jaw, and then the upper appliance wings are screwed or fastened into position using fasteners. Then, the matingly angled surfaces 106, 130 of the upper and lower wings contact and force the lower appliance to laterally shift or become offset with respect to the upper appliance. The lower jaw then protrudes beyond the upper jaw in a lateral direction (to the right in FIG. 10).

In existing appliances, tabs on the left and right of the jaws prevent lateral shifting of the jaws. An advantage of the disclosure is independent lateral movement of the upper and lower mandible. Some existing devices lock the appliances together so there is no independent lateral movement. The adjustable fang or post on the lower appliance allows the lower jaw to be advanced and allows independent opening and closing of the mouth.

Air channels can be incorporated in temporary or permanent appliances to provide room air or oxygen to the user. The hose or tube can be easily attached or removed.

Another advantage of the appliance is that it can be temporary or custom fitted.

The exemplary embodiment has been described with reference to the preferred embodiments. Obviously, modifications and alterations will occur to others upon reading and understanding the preceding detailed description. It is intended that the exemplary embodiment and the appended claims be construed as including all such modifications and alterations.

1. A mandibular advancement splint which provides for adjustable lateral movement of upper and lower appliances, comprising:
an upper appliance which is inserted onto a user's upper jaw;
a lower appliance which is inserted onto a user's lower jaw;
a first protrusion formed on said upper appliance;
a second protrusion formed on said lower appliance, wherein one of said first protrusion and said second protrusion can be adjusted to offset said lower jaw with respect to said upper jaw.

2. The splint of claim 1, wherein said second protrusion comprises a body having an opening therein and a shim which extends through said opening to adjust the thickness of the protrusion.

3. The splint of claim 2, wherein said shim comprises thermoplastic dental material.

4. The splint of claim 3, wherein said shim is substantially T-shaped.

5. The splint of claim 1, wherein said upper appliance and said lower appliance comprise thermoplastic dental material.

6. The splint of claim 1, wherein said first protrusion is integrally formed with said upper appliance.

7. The splint of claim 1, wherein said first protrusion is attached as a separate piece to said upper appliance.

8. The splint of claim 1, wherein when said second protrusion engages said first protrusion, said second protrusion slides along a surface of said first protrusion which moves said lower jaw laterally outwardly with respect to said upper jaw.

9. The splint of claim 1, wherein said second protrusion comprises a channel for accommodating an associated air supply connector.

10. The splint of claim 1, wherein at least one of said upper appliance and said lower appliance comprises a channel for receiving deformable material.

11. The splint of claim 1, further comprising an air supply tube connected to at least one of said upper appliance and said lower appliance.

12. The splint of claim 1, wherein at least one of said upper and lower appliances comprises a tray for holding deformable material.

13. The splint of claim 1, wherein at least one of said upper appliance and said lower appliance comprises a tray custom fitted to a user's upper or lower jaw.

14. The splint of claim 12, wherein said tray is made of acrylic or plastic material.

15. The splint of claim 13, wherein said tray is made of acrylic or plastic material.

16. The splint of claim 1, further comprising a viscoelastic energy absorbing material mounted adjacent at least one of said upper and lower appliances.

17. A mandibular advancement splint, comprising:
an upper appliance having a body mounted to an upper jaw of a user;
a lower appliance having a body mounted to a lower jaw of a user;
wherein said upper appliance comprises a pair of wings attached to said upper appliance body;
said lower appliance comprises a pair of wings attached to said lower appliance body;
wherein said pair of wings attached to said upper appliance are slidably adjustable with respect to said upper appliance body to determine the offset between the upper and lower jaws or a user.

18. The splint of claim 17, wherein said upper appliance body comprises a pair of channels formed in side walls of the body of said upper appliance.

19. The splint of claim 18, wherein said first wings each comprises at least one opening for receiving a fastener to secure said first wings to said respective channels of said upper appliance.

20. The splint of claim 19, wherein each of said first wings has an angled front wall.

21. The splint of claim 20, wherein each of said second wings has an angled rear wall which matingly engages said angled front wall of said first wings to offset said lower appliance body with respect to said upper appliance body.

22. The splint of claim 21, wherein said second wings are fixedly attached to said lower appliance body.

23. The splint of claim 21, wherein said second wings are integrally formed with said lower appliance body.

24. The splint of claim 17, wherein each of said upper appliance body and said lower appliance body has a shim placed at a posterior portion of said upper appliance body and said lower appliance body to facilitate movement of a user's tongue.

25. The splint of claim 17, wherein each of said upper appliance body and said lower appliance body comprises a groove for receiving deformable material.

26. The splint of claim 17, wherein said lower appliance body comprise a notch formed centrally therein.
